(19) United States
(12) Patent Application Publication
(10) Pub. No.: US 2014/0256031 A1
(43) Pub. Date: Sep. 11, 2014

(54) SEMICONDUCTOR MICRO-ANALYSIS CHIP AND SAMPLE LIQUID FLOWING METHOD

(71) Applicant: KABUSHIKI KAISHA TOSHIBA, Tokyo (JP)

(72) Inventors: Kentaro KOBAYASHI, Tokyo (JP); Hiroko MIKI, Yokohama-shi (JP); Akihiro KOJIMA, Yokohama-shi (JP); Hideto FURUYAMA, Yokohama-shi (JP)

(73) Assignee: KABUSHIKI KAISHA TOSHIBA, Toyko (JP)

(21) Appl. No.: 14/011,640

(22) Filed: Aug. 27, 2013

(30) Foreign Application Priority Data
Mar. 7, 2013 (JP) 2013-045395

(51) Int. Cl. G01N 33/487

(52) U.S. Cl. CPC G01N 33/4875
USPC 435/287.3

(57) ABSTRACT

According to one embodiment, a semiconductor micro-analysis chip for detecting fine particles in sample liquid includes a semiconductor substrate, a flow channel formed in the semiconductor substrate and having a sample liquid inlet and sample liquid outlet at end portions thereof, and an absorber provided on at least part of the sample outlet of the flow channel to absorb the sample liquid.

### CROSS-REFERENCE TO RELATED APPLICATIONS

This application is based upon and claims the benefit of priority from Japanese Patent Application No. 2013-045395, filed Mar. 7, 2013, the entire contents of which are incorporated herein by reference.

### FIELD

Embodiments described herein relate generally to a semiconductor micro-analysis chip for detecting a fine particle sample and a sample liquid flowing method.

### BACKGROUND

In the field of biotechnology or health care, much attention is given to a micro-analysis chip having microfluidic devices such as a micro-flow channels and detection systems integrated therein. These micro-analysis chips are mainly made of glass substrates. In most cases, a flow channel formed in the glass substrate is capped by bonding a cover glass plate thereon. As sample detection techniques, laser light scattering detection and fluorescent detection is often utilized.

However, if a glass substrate is used, it is difficult to form a minute structure. Further, it is necessary to form a lid of the flow channel by bonding the substrate thereon, which leads to difficulty in mass production of the devices. Therefore, there is a problem that the cost reduction is difficult.

### BRIEF DESCRIPTION OF THE DRAWINGS

FIG. 1A is a plan view showing the schematic configuration of a semiconductor micro-analysis chip according to a first embodiment.

FIG. 1B is a cross-sectional view showing the schematic configuration of the semiconductor micro-analysis chip according to the first embodiment.

FIGS. 2A, 2B are schematic views showing one example of a particle detector used in the analysis chip of FIGS. 1A, 1B.

FIG. 3A is a plan view showing the schematic configuration of a semiconductor micro-analysis chip according to a second embodiment.

FIG. 3B is a cross-sectional view showing the schematic configuration of the semiconductor micro-analysis chip according to the second embodiment.

FIGS. 4A to 4D are cross-sectional views showing a manufacturing process of a pillar array used in the analysis chip of FIGS. 3A, 3B.

FIGS. 5A to 5D are plan views showing the placement of absorbers.

### DETAILED DESCRIPTION

In general, according to one embodiment, a semiconductor micro-analysis chip for detecting fine particles in sample liquid comprises a semiconductor substrate, a flow channel formed in the semiconductor substrate and having a sample liquid inlet and sample liquid outlet at end portions thereof, and an absorber provided on at least part of the sample liquid outlet of the flow channel to absorb the sample liquid.

Embodiments are explained with reference to the drawings. In this case, several concrete materials and configurations are taken as examples and explained, but other materials and configurations having the same functions can be used. Therefore, this invention is not limited to the following embodiments.

### First Embodiment

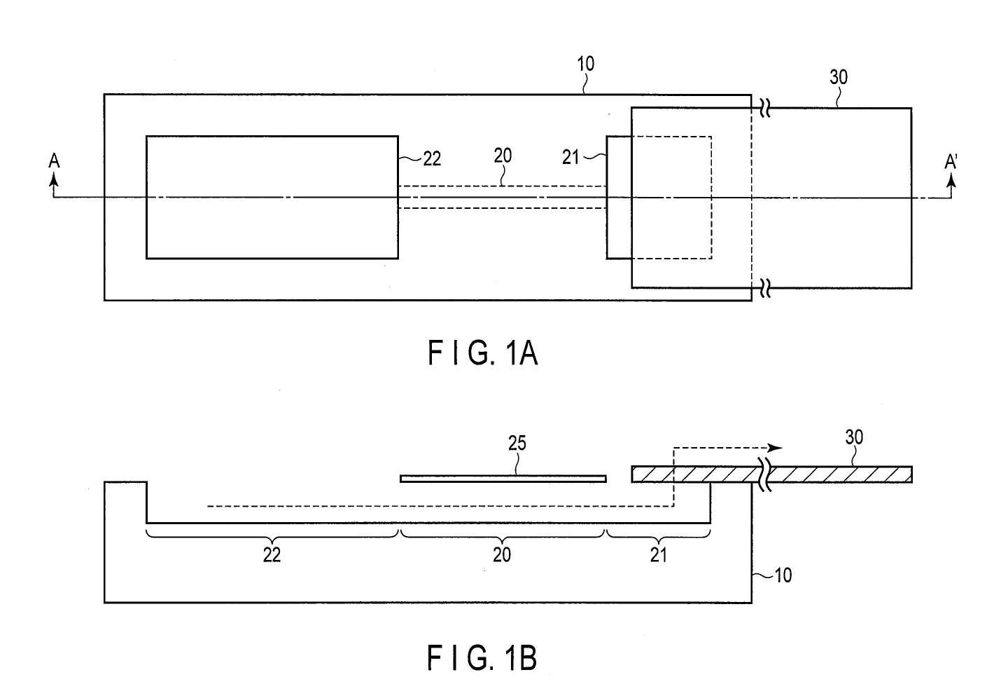

FIG. 1A is a plan view showing the schematic configuration of a semiconductor micro-analysis chip according to a first embodiment and FIG. 1B is a cross-sectional view taken along line A-A′ of FIG. 1A.

In FIGS. 1A, 1B, a flow channel 20 formed of a linear groove is formed in the surface portion of a Si substrate (semiconductor substrate) 10. The flow channel 20 is formed by linearly etching the surface portion of the Si substrate 10 and the upper surface thereof is covered with a cap layer 25 to form a capped flow channel. Further, both ends of the flow channel 20 are widened to form fluid reservoir used for injecting and discharging sample liquid. That is, a sample liquid outlet 21 is provided at one end portion of the flow channel 20 and a sample liquid inlet 22 is provided at the other end portion.

The sample liquid inlet 22 is formed to have a larger area than the sample liquid outlet 21. Further, the wall surfaces and bottom surfaces of the flow channel 20, sample liquid outlet 21 and sample liquid inlet 22 may be formed of Si itself or may be formed of SiO₂ obtained by oxidizing Si.

The capped flow channel 20 can be formed by use of the following method. That is, a sacrifice layer such as an organic coating film or the like is formed on an Si substrate 10 having a groove formed therein by etching, the sacrifice layer is made flat with reducing the film thickness thereof by use of the etch-back or CMP (Chemical Mechanical Polishing) technique, and consequently the sacrifice layer is embedded only in the groove. Then, an oxide film or the like used as a cap layer 25 is formed on the structure. Next, portions of the cap layer 25 that lie on the sample liquid inlet 22 and sample liquid outlet 21 are removed by use of the photolithography technique and etching technique and the sacrifice layer is finally removed by oxygen plasma asking or the like to form a capped flow channel 20.

An absorber 30 capable of absorbing sample liquid is placed on the sample liquid outlet 21. As the absorber 30, for example, a fiber assembly of filter paper, nonwoven fabric or the like can be used. The absorber 30 may be placed to cover a portion or the whole portion of the sample liquid outlet 21.

With the above configuration, if sample liquid containing to-be-detected fine particles is dropped onto the sample liquid inlet 22, the sample liquid is drawn into the capped flow channel 20 by capillary action and then the sample liquid reaches the sample liquid outlet 21 via the flow channel 20. Then the sample liquid is absorbed by means of the absorber 30 provided on the sample liquid outlet 21. When the sample liquid in the sample liquid outlet 21 once starts to be absorbed by means of the absorber 30, the following sample liquid successively absorbed by means of the absorber 30, and therefore, the sample liquid in the flow channel 20 continuously flows. That is, by absorbing the sample liquid using the absorber 30, the sample liquid in the flow channel 20 can flow without using electrophoresis or external pump. Then, the fine particles contained in the sample liquid can also be moved according to the flow of the sample liquid.

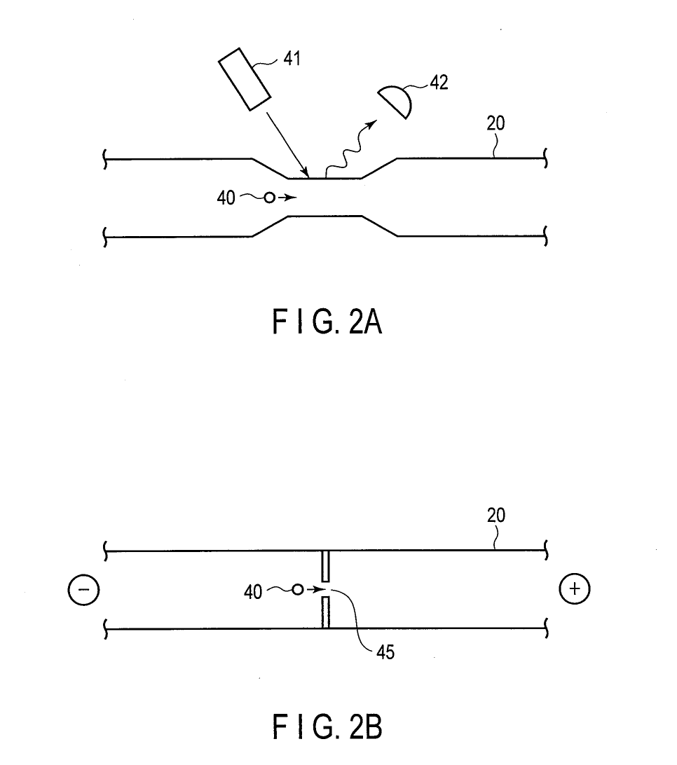

To detect fine particles in sample liquid flowing through the flow channel 20, several methods such as, for example, observing scattered light from the fine particles caused by irradiating laser light or observing variation of the ion current by use of a nano-hole can be used. As shown in FIG. 2A, with the method based on the laser light application, laser light from a laser source 41 is irradiated to the fine particles 40 flowing in the flow channel 20, and the scattered light from the fine particles 40 are detected on a detector 42.

Further, in the method using a nano-hole, as shown in FIG. 2B, a nano-hole (fine hole) 45 is formed inside the flow channel 20, and a voltage is applied between electrodes inserted on the upstream side and downstream side of the nano-hole 45. Using a conductive liquid such as an electrolytic solution as the sample liquid, ion current flows through the flow channel 20 filled with the sample liquid. When the fine particles 40 pass through the nano-hole 45, the ion current varies according to the diameter of the fine particles 40, and thus the diameter of the fine particles 40 can be measured by measuring the ion current variation.

According to the present embodiment, since the absorber 30 is placed in contact with the sample liquid outlet 21 of the capped flow channel 20, the sample liquid is absorbed by the absorber 30 and, as a result, flow of the sample liquid in the flow channel 20 is caused. Therefore, the fine particles in the sample liquid can move with the flow without using electrophoresis or the like. Furthermore, since the present embodiment can be realized with a very simple configuration obtained by forming an etching groove in the surface portion of the Si substrate 10 and placing the absorber 30, the structural body used to detect fine particles can be realized at low cost.

This leads to realization of a small and mass-productive semiconductor micro-analysis chip at low cost, which can detect a virus, bacteria, or the like with high sensitivity.

### Second Embodiment

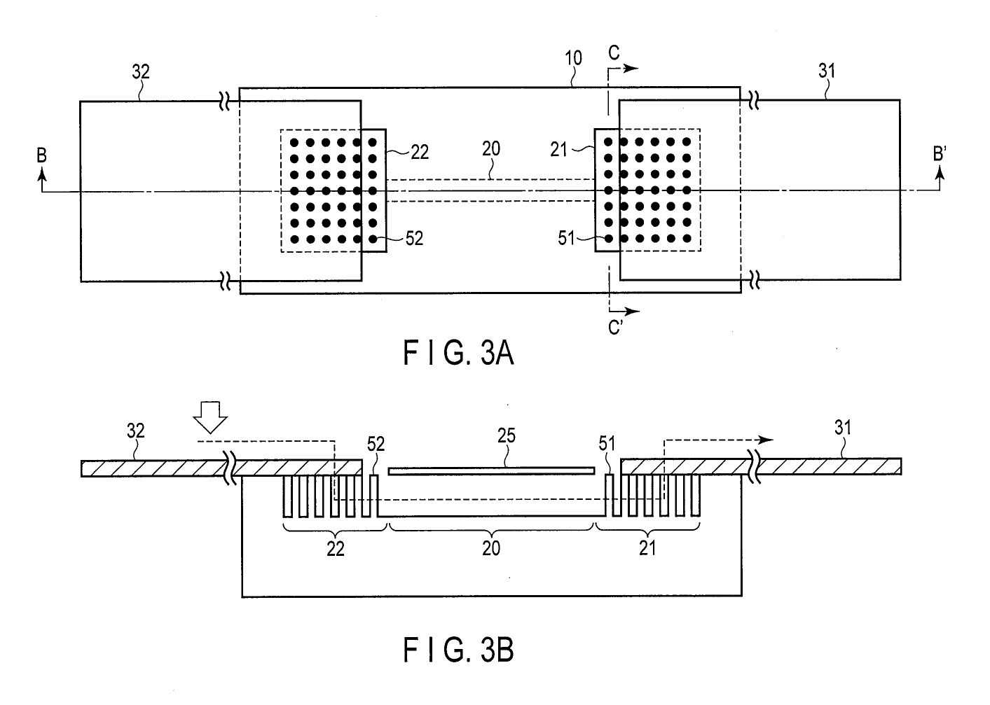

FIG. 3A is a plan view showing the schematic configuration of a semiconductor micro-analysis chip according to a second embodiment and FIG. 3B is a cross-sectional view taken along line B-B′ of FIG. 3A. Portions that are the same as those of FIGS. 1A, 1B are denoted by the same symbols and the detailed explanation thereof is omitted.

The present embodiment is different from the first embodiment explained before in that absorbers are provided on both the sample liquid outlet 21 and sample liquid inlet 22 of the flow channel 20 and that columnar structures (pillars) are provided on the sample liquid outlet 21 and on the sample liquid inlet 22 of the flow channel 20. In this case, a pillar array (nano-pillars) 51 consisted of a columnar structure group is provided on the sample liquid outlet 21 which is formed on one end portion of the capped flow channel 20 and a pillar array 52 is provided on the sample liquid inlet 22 which is formed on the other end portion thereof. The pillar arrays 51 and 52 are the pillars placed at regulation distance on the sample liquid outlet 21 and the sample liquid inlet 22, respectively, and are obtained by etching a Si substrate 10. When etching masks for forming the flow channel 20, the sample liquid outlet 21 and the sample liquid inlet 22 by etching the Si substrate 10 are formed, etching masks for the pillar arrays 51, 52 are formed at the same time. Then using the above masks, the pillar arrays 51, 52 are formed by reactive ion etching (RIE) of the Si substrate 10.

The pillar arrays 51, 52 may be pure Si, Si with partially-oxidized surface, or SiO2. Further, pillar arrays can be arranged not only on the sample liquid outlet 21 and sample liquid inlet 22 but also in the flow channel 20.

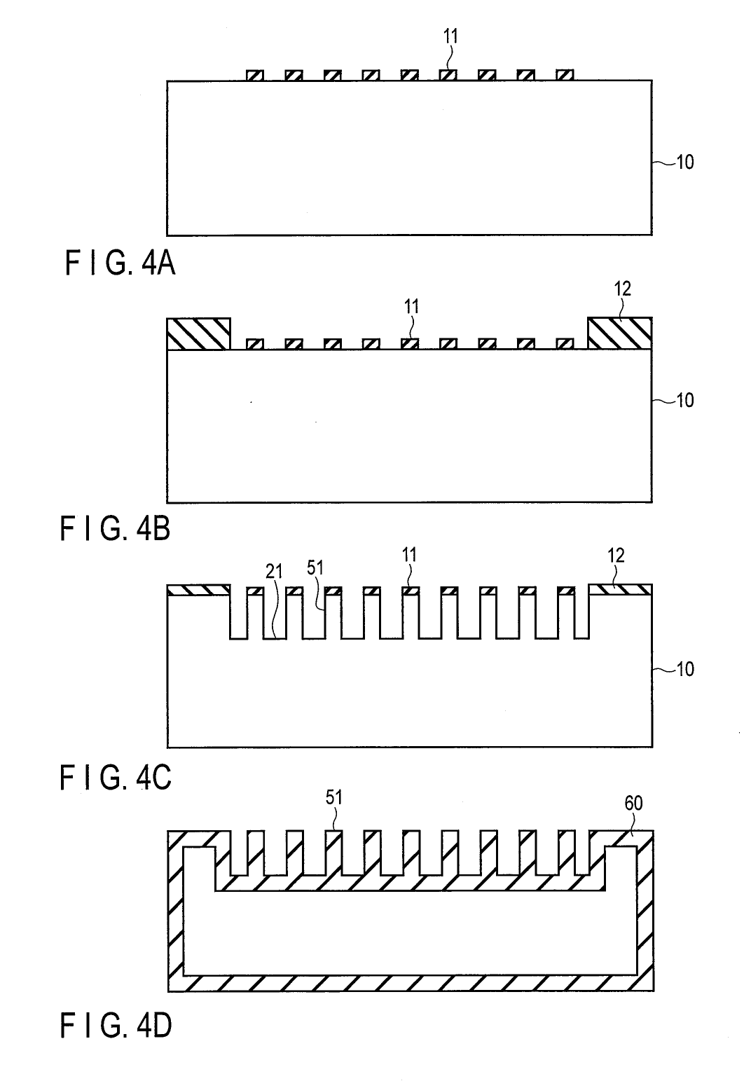

One example of the manufacturing method of the pillar array 51 is explained with reference to FIGS. 4A to 4D. FIGS. 4A to 4D correspond to the cross-sections taken along line C-C′ of FIG. 3A and shows a portion of the sample liquid outlet 21. A portion of the sample liquid inlet 22 also has the same configuration.

First, as shown in FIG. 4A, an etching mask 11 for a pillar array is formed on a Si substrate (a semiconductor substrate) 10. For example, the etching mask 11 is obtained by forming a SiO₂ film on the Si substrate 10, forming thereon a resist mask pattern of the pillar array, and etching the SiO₂ film with the resist mask.

Next, as shown in FIG. 4B, the etching masks 12 for forming the flow channel and other regions are formed. As is the case of the etching mask 11, the etching mask 12 is also obtained by forming a resist pattern and etching. The etching mask 12 may be formed at the same time as formation of the etching mask 11 for the pillar array depicted in FIG. 4A.

Then, as shown in FIG. 4C, pillar array 51 and the sample liquid outlet 21 of the flow channel 20 are formed by etching the Si substrate 10 by RIE or the like using the etching masks 11, 12.

Next, the etching masks 11, 12 are removed, and an oxidation process is performed to oxidize the whole portion of pillar array 51 as shown in FIG. 4D. As a result, the pillar array 51 turns from Si to SiO₂, and the exposed surface portion of the Si substrate 10 is covered with an oxide film 60.

In addition, in this embodiment, as shown in FIG. 3B, a first absorber 31 is arranged to make contact with the pillar array 51 of the sample liquid outlet 21. Likewise, a second absorber 32 is arranged to make contact with the pillar array 52 of the sample liquid inlet 22.

In this case, it is necessary to consider the following points when the pillar array 51 in the sample liquid outlet 21 is oxidized. It is known that the molar volumes of Si and SiO₂ are 12.06 and 27.20 cm³, respectively, and when SiO₂ is formed by thermal oxidization of Si, the volume expands to 2.26 times. That is, when the pillar surface is thermally oxidized, the pillar diameter/interval varies from the configuration obtained after the Si substrate is etched. Further, if the oxidation rate for each Si pillar is uneven, the diameters and intervals of the pillars will be varied.

Meanwhile, if the thermal oxidation process is performed until the pillars completely turn into SiO₂, the diameter of the pillar does not become larger than a certain value. Since the volume ratio of Si and SiO₂ is already known as described above, the amount of size change associated with the change of the pillar from Si to SiO2 completely can be previously estimated. In this way, the pillar diameter/interval can be easily controlled by sufficiently oxidizing the Si substrate surface and completely oxidizing the pillars in the manufacturing method of this embodiment.

With the above configuration, sample liquid is dropped onto the absorber 32. The sample liquid oozed from the absorber 32 passes into the flow channel 20 via a portion of the sample liquid inlet 22 having excellent wettability. When no pillar array 52 is provided, the sample liquid of the absorber 32 is often prevented from passing into the flow channel 20 by the presence of a space between the flow channel 20 and the absorber 32. However, if there is the pillar array 52 in contact with the absorber 32, the sample liquid is absorbed because of the surface tension in the spaces between the pillars of the pillar array 52. Therefore, the sample liquid is smoothly introduced from the absorber 32 into the sample liquid inlet 22 and capped flow channel 20 via pillar array 52.

In addition, the sample liquid flowing through the capped flow channel 20 is gradually absorbed into the absorber 31 via the pillar array 51 on the side of the sample liquid outlet 21. By absorbing the sample liquid with the absorber 31, the sample liquid in the flow channel 20 is drawn and is facilitated to flow. Therefore, the sample liquid and fine particles in the flow channel 20 can be caused to flow without using electrophoresis.

Further, using the absorber 32 on the sample liquid inlet 22, a sufficiently large amount of the sample liquid can be supplied to the flow channel 20 without increasing the size of the semiconductor micro-analysis chip. Generally, sample liquid is injected into the micro-analysis chip using a micropipette or the like and the drop amount is approximately 10 to 10000 μL. In order to receive the sample liquid of this amount, for example, an area of 100 mm² with a depth of 100 μm is needed. If the reception region is integrated on the micro-analysis chip, the chip size becomes extremely larger than the size required for integrating a functional portion as an analysis chip, and then the cost increases extremely. In addition, the concentration of fine particles in the sample liquid is generally low and in case a large number of fine particles are needed for detection, a large amount of sample liquid must be injected, resulting in the huge size of the sample liquid inlet 22.

In this embodiment of the semiconductor micro-analysis chip, a sufficiently large absorber 32 is provided on the exterior of the analysis chip instead of integrating the extremely large sample liquid inlet, and sample liquid is dropped onto the absorber 32 and injected into the flow channel 20. Further, sample liquid discharged from the sample liquid outlet 21 can be absorbed by the absorber 31. As a result, sample liquid of an amount larger than the amount of sample liquid treated in the analysis chip can be injected and discharged.

Thus, in this embodiment, a large amount of sample liquid can be treated even with an extremely small analysis chip and functional portions of a semiconductor micro-analysis chip can be integrated in a minimum area. Therefore, the cost can be markedly reduced.

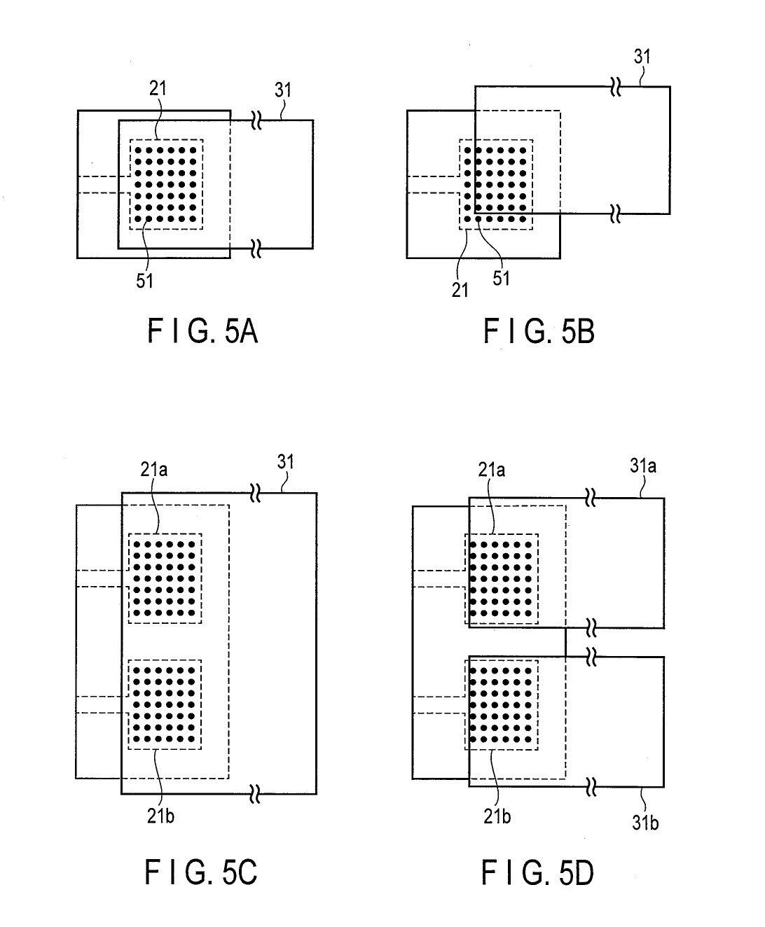

As the arrangement method of the absorber 31, the absorber 31 may be arranged to cover the whole portion of the sample liquid outlet 21 as shown in FIG. 5A, or to cover a portion of the sample liquid outlet 21 as shown in FIG. 5B. Further, as shown in FIG. 5C, the absorber 31 may be arranged to simultaneously cover the sample liquid outlets 21a, 21b of plural flow channels. Additionally, as shown in FIG. 5D, different absorbers 31a, 31b may be arranged to cover sample liquid outlets 21a, 21b of plural flow channels. Also, the absorber 32 on the side of the sample liquid inlet 22 can be arranged in the same manner as described above.

According to the present embodiment, the same effect as that of the first embodiment can be attained and the following effect can be obtained by arranging the absorber 31 to cover the sample liquid outlet 21 and arranging the absorber 32 to cover the sample liquid inlet 22.

That is, a sufficiently large amount of sample liquid can be injected in the flow channel 20 without increasing the size of the semiconductor micro-analysis chip by providing not only the absorber 31 on the side of the sample liquid outlet 21 but also the absorber 32 on the side of the sample liquid inlet 22. That is, the semiconductor micro-analysis chip can be further miniaturized.

Further, sample liquid can be more easily transmitted from the sample liquid outlet 21 to the absorber 31 by providing pillar array 51 and sample liquid can be more easily transmitted from the absorber 32 to the sample liquid inlet 22 by providing pillar array 52. That is, the continuity between the flow channel 20 and the absorbers 31, 32 can be enhanced by fully laying the pillars at predetermined diameters and intervals in the sample liquid outlet 21 and sample liquid inlet 22.

Further, since the undersurfaces of the absorbers 31, 32 can be supported by the pillar arrays 51, 52, it becomes advantageous in structure.

(Modification)

This invention is not limited to the above embodiments.

In the embodiments, the Si substrate is used as the semiconductor substrate, but the substrate is not limited to Si and another semiconductor can be used if a groove and pillars can be formed by use of a normal semiconductor manufacturing process.

The particle detection mechanism is not limited to the case shown in FIGS. 2A, 2B and can be adequately modified according to the specification. As the material of the absorber, a material that can adequately absorb sample liquid can be used and can be adequately modified according to the specification.

In the embodiments, the cap layer is provided to cover the flow channel, but the cap layer is not necessarily required and the structure can be applied to an open type nano-pillar laying flow channel. Further, the placement position of the absorber is not limited to the portion on the sample liquid outlet and, for example, the structure can be applied to a type in which the sample liquid outlet laterally presses the absorber in an end face direction.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A semiconductor micro-analysis chip for detecting fine particles in sample liquid, comprising:
a semiconductor substrate,
a flow channel provided in the semiconductor substrate and having a sample liquid inlet provided on one end side of the flow channel and a sample liquid outlet provided on the other end side, and
a first absorber provided on at least part of the sample liquid outlet of the flow channel, the absorber being configured to absorb the sample liquid.

2. The chip according to claim 1, wherein the flow channel includes a groove formed in a surface portion of the semiconductor substrate and a cap layer formed to cover the groove in a region other than the sample liquid inlet and the sample liquid outlet.

3. The chip according to claim 1, wherein a pillar array formed of columnar structures is arranged in the sample liquid outlet and the pillar array in the sample liquid outlet and the first absorber are arranged at a close distance to transfer the sample liquid or arranged in contact with each other.

4. The chip according to claim 3, wherein the pillar array is formed of one of the semiconductor substrate, an oxide of the semiconductor substrate or a composite material thereof.

5. The chip according to claim 1, wherein the sample liquid inlet has an area larger than that of the sample liquid outlet.

6. The chip according to claim 1, further comprising a second absorber provided on at least part of the sample liquid inlet and the second absorber absorbs the sample liquid.

7. The chip according to claim 6, wherein pillar arrays formed of columnar structures are arranged in the sample liquid inlet and in the sample liquid outlet, where the pillar array in the sample liquid outlet and the first absorber, and the pillar array in the sample liquid inlet and the second absorber are arranged at a close distance to transfer the sample liquid or arranged in contact with each other, respectively.

8. The chip according to claim 7, wherein the pillar array is formed of one of the semiconductor substrate, an oxide of the semiconductor substrate or a composite material thereof.

9. The chip according to claim 1, further comprising a detection mechanism configured to detect fine particles in the sample liquid flowing in the flow channel by applying laser light.

10. The chip according to claim 1, further comprising a member having a nano-hole (fine hole) formed therein and arranged in an intermediate portion of the flow channel and a detection mechanism configured to measure an ion current variation caused when the fine particles pass through the nano-hole.

11. A semiconductor micro-analysis chip for detecting fine particles in sample liquid, comprising:
a semiconductor substrate,
a flow channel provided in the semiconductor substrate, having a sample liquid inlet and a sample liquid outlet provided on each end portion thereof and covered with a cap layer,
pillar arrays formed of columnar structures arranged in the sample liquid inlet and in the sample liquid outlet,
a first absorber provided on at least part of the sample liquid outlet of the flow channel, the first absorber being arranged at a close distance with respect to the pillar array in the sample liquid outlet to transfer the sample liquid or arranged in contact with the pillar array and being configured to absorb the sample liquid, and
a second absorber provided on at least part of the sample liquid inlet of the flow channel, the second absorber being arranged at a close distance with respect to the pillar array in the sample liquid inlet to transfer the sample liquid or arranged in contact with the pillar array and being configured to absorb the sample liquid.

12. The chip according to claim 11, wherein the pillar array is formed of one of the semiconductor substrate, an oxide of the semiconductor substrate or a composite material thereof.

13. The chip according to claim 11, further comprising a detection mechanism configured to detect fine particles in the sample liquid flowing in the flow channel by applying laser light.

14. The chip according to claim 11, further comprising a member having a nano-hole (fine hole) formed therein and arranged in an intermediate portion of the flow channel and a detection mechanism configured to measure an ion current variation caused when the fine particles pass through the nano-hole.

15. A sample liquid flowing method, comprising:
supplying a sample liquid into a sample liquid inlet provided on one end side of a flow channel formed in a semiconductor substrate,
discharging the sample liquid from a sample liquid outlet provided on the other end side of the flow channel,
arranging a first absorber in contact with at least part of the sample liquid outlet, and
causing the first absorber to absorb the sample liquid flowing through the flow channel.

16. The sample liquid flowing method according to claim 15, further comprising:
arranging a second absorber in contact with at least part of the sample liquid inlet,
causing the second absorber to absorb the sample liquid supplied into the sample liquid inlet, and
injecting the sample liquid oozing from the sample liquid inlet to the flow channel.
